(19) United States
(12) Patent Application Publication
(10) Pub. No.: US 2014/0200635 A1
(43) Pub. Date: Jul. 17, 2014

(54) PHOTOTHERAPY DEVICE

(71) Applicants: Yoshiteru II, Kanagawa (JP); Yuki Kawase, Kanagawa (JP); Daisuke Okamura, Kanagawa (JP); Shuhei Tanaka, Kanagawa (JP)

(72) Inventors: Yoshiteru II, Kanagawa (JP); Yuki Kawase, Kanagawa (JP); Daisuke Okamura, Kanagawa (JP); Shuhei Tanaka, Kanagawa (JP)

(73) Assignee: PANASONIC CORPORATION, Osaka (JP)

(21) Appl. No.: 14/239,296

(22) PCT Filed: Aug. 20, 2012
(86) PCT No.: PCT/JP2012/005207

§ 371 Date: Feb. 18, 2014

(30) Foreign Application Priority Data
Aug. 22, 2011 (JP) 2011-180156

(51) Int. Cl. A61N 5/06

(52) U.S. Cl. CPC A61N 5/0613
USPC 607/88

(57) ABSTRACT

Provided is a phototherapy device which enables a sufficient amount of light to reach a deep tissue to achieve a satisfactory therapy while preventing the increase in the skin temperature of an affected part, and which can achieve a comfortable therapy. The present invention provides a phototherapy device comprising a sheet-like member having a first surface on which an affected part is to be arranged and a surface light source for emitting light toward the sheet-like member, wherein the first surface is formed in an uneven shape so as to provide multiple grooves. The phototherapy device can be used for, for example, the treatment of an affected part in a rheumatoid arthritis patient.

### TECHNICAL FIELD

The present invention relates to a phototherapy apparatus.

### BACKGROUND ART

Phototherapy apparatuses are used for relief of pain due to chronic non-infectious inflammation of muscles or joints or for treatment of rheumatoid arthritis, by applying light to the affected part such as hand, wrist, foot, ankle or knee. Rheumatoid arthritis occurs for example over the entire joints of a hand such as metacarpophalangeal joints and/or proximal interphalangeal joints. Thus, there are cases wherein higher therapeutic efficiencies can be achieved when using, as a light source for a phototherapy apparatus for treating rheumatoid arthritis, a surface light source that can apply light to the entire phalangeal joints than when using a light source that locally applies light, such as a laser light source.

As a phototherapy apparatus using such a surface light source, a phototherapy apparatus has been proposed that allows an affected part to be directly touched to the surface light source, although such a phototherapy apparatus is not intended for treatment of rheumatoid arthritis (see, for example, PTL 1).

### CITATION LIST

### Patent Literature

### PTL 1

Japanese Patent Application Laid-Open No. 11-192315

### SUMMARY OF INVENTION

### Technical Problem

In treatment using a phototherapy apparatus including a surface light source, when an affected part is directly touched to the surface light source, the temperature of the skin increases, which may cause redness and/or low temperature burn of the affected part. In phototherapy, depending on the symptom of an affected part to be treated, it is necessary to deliver light to a deep tissue in the affected part. In order to deliver light to a deep tissue, it is necessary to apply high-power light to the affected part. When high-power light is applied, redness and/or low temperature burn of the affected part is more likely to occur.

In particular, in the case of treatment of, e.g., rheumatoid arthritis, compared to cases of treatment of, e.g., shoulder stiffness or low back pain, it is more necessary to deliver light to a deep tissue and thus apply high-power light from a surface light source to the affected part. Therefore, redness or low temperature burn of the affected part due to an increase in temperature of the skin is more likely to occur.

Also, when an affected part is directly touched to a surface light source and light from, e.g., the surface light source is applied to a part that is touched to the affected part and an area around that part, heat easily persists in the affected part. Therefore, when light is applied to the affected part for a certain period of time, the affected part and the part around the affected part easily sweat, providing a feeling of discomfort.

On the other hand, if low-power light is applied to an affected part in order to limit an increase in the skin temperature of the affected part, a long period of time may be required for treatment and/or the light is not delivered to the deep tissue resulting in failure of sufficient treatment.

Therefore, an object of the present invention is to provide a phototherapy apparatus that, while limiting an increase in the skin temperature of the affected part, delivers sufficient light to a deep tissue in the affected part, enabling sufficient and comfortable treatment.

### Solution to Problem

To achieve the above object, a phototherapy apparatus according to the present invention includes a sheet-like member including a first surface on which an affected part is to be rested, and a surface light source configured to apply light toward the sheet-like member, wherein a plurality of grooves are provided in the first surface by forming thereon irregularities.

### Advantageous Effects of Invention

According to the phototherapy apparatus according to the present invention having such a configuration as described above, it is possible to deliver sufficient light to a deep tissue in the affected part while limiting an increase in the skin temperature of an affected part, thus enabling sufficient and comfortable treatment.

### BRIEF DESCRIPTION OF DRAWINGS

FIG. 1 is a perspective diagram of a phototherapy apparatus according to Embodiment 1 of the present invention;

FIG. 2 is a perspective diagram illustrating the use state of the phototherapy apparatus according to Embodiment 1 of the present invention;

FIG. 3 is a transparent perspective view of the phototherapy apparatus according to Embodiment 1 of the present invention, illustrating an affected part insertion room;

FIG. 4 is a side cross-sectional view of the phototherapy apparatus according to Embodiment 1 of the present invention, illustrating the affected part insertion room;

FIG. 5 is a top cross-sectional view of the phototherapy apparatus according to Embodiment 1 of the present invention, illustrating the affected part insertion room;

FIG. 6 is a side cross-sectional view of the phototherapy apparatus according to Embodiment 1 of the present invention, illustrating the use state of the apparatus;

FIG. 7 is a perspective view of a sheet-like member in the phototherapy apparatus according to Embodiment 1 of the present invention;

FIG. 8 is a top view of a first surface light source and a second surface light source in the phototherapy apparatus according to Embodiment 1 of the present invention;

FIGS. 9-A1 and 9-A2 are conceptual diagrams illustrating phototherapy using a conventional phototherapy apparatus, and FIGS. 9-B1 and 9-B2 are conceptual diagrams of phototherapy using the phototherapy apparatus according to Embodiment 1 of the present invention;

FIG. 10 is a top cross-sectional view of a phototherapy apparatus according to Embodiment 2 of the present invention, illustrating an affected part insertion room;

FIG. 11 is a perspective view illustrating protrusions formed on a first surface in the phototherapy apparatus according to Embodiment 2 of the present invention;

FIG. 12 is a top cross-sectional view of the phototherapy apparatus according to Embodiment 2 of the present invention, illustrating a state in which an affected part is rested on the protrusions formed on the first surface;

FIG. 13 is a diagram illustrating an example of the protrusions formed on the first surface in the phototherapy apparatus according to Embodiment 2 of the present invention; and

FIG. 14 is a diagram illustrating an example of the protrusions formed on the first surface in the phototherapy apparatus according to Embodiment 2 of the present invention.

### DESCRIPTION OF EMBODIMENTS

A phototherapy apparatus according to the present invention includes a sheet-like member including a first surface on which an affected part is to be rested, and a surface light source configured to apply light toward the sheet-like member.

The sheet-like member is made of a material that transmits light emitted from the surface light source. As described later, light from the surface light source is preferably infrared light, and in such case, the sheet-like member is made of, for example, a transparent acrylic resin or transparent glass. The sheet-like member includes a first surface (affected part resting surface) on which an affected part is to be rested, and a second surface that is a surface opposite to the first surface.

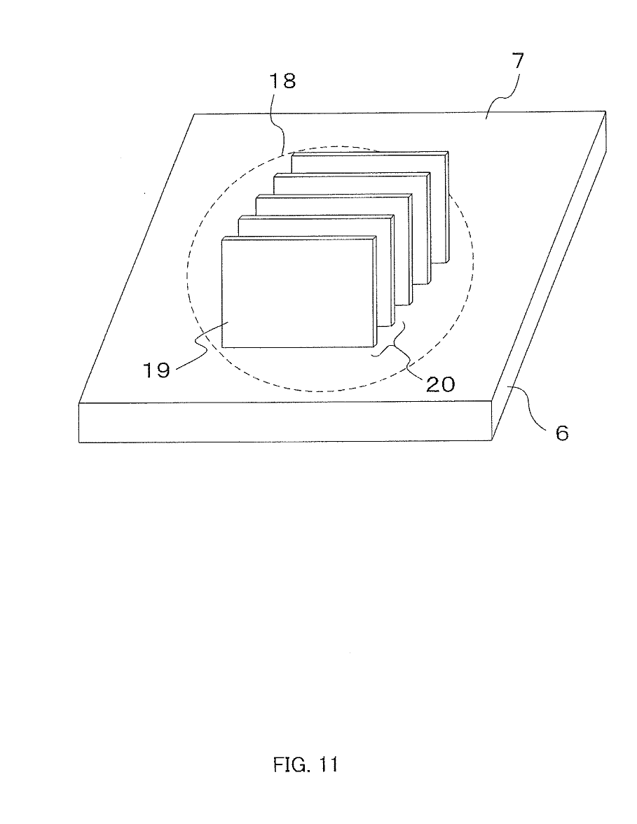

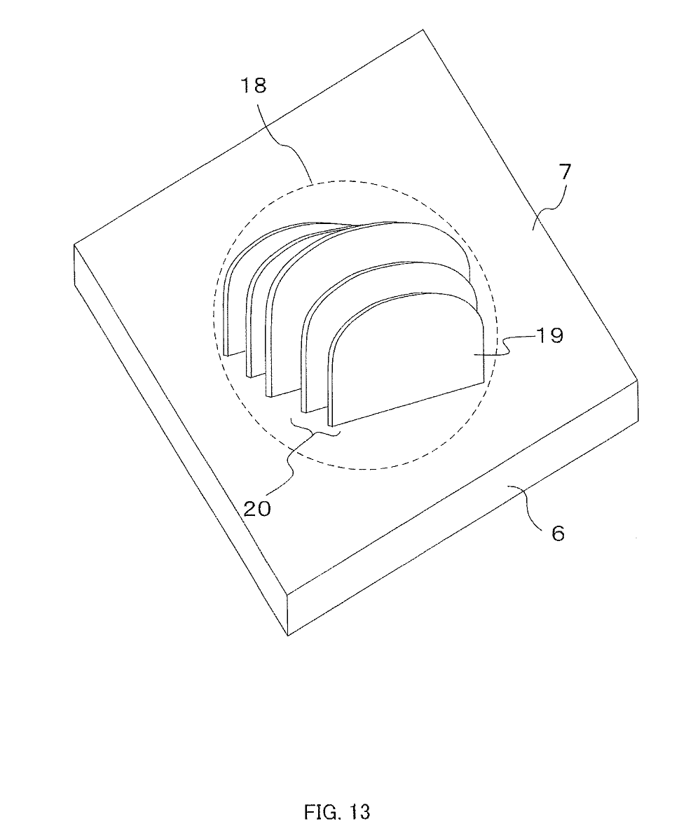

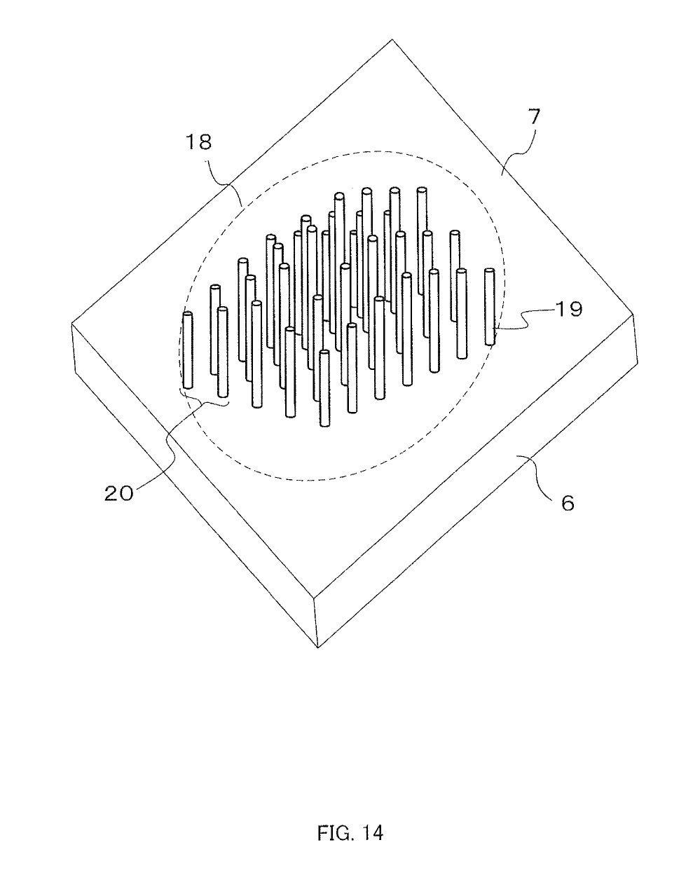

The first surface of the sheet-like member includes irregularities. The irregularities may be formed over the entire first surface of the sheet-like member (see Embodiment 1) or may alternatively be formed at least in an area in which an affected part is to be rested (affected part resting area) (see Embodiment 2). The irregularities are fainted of, for example, a plurality of mutually-parallel flat plate-like protruding objects (see FIG. 11 or 13) or a plurality of rod-like protruding objects (see FIG. 14). As illustrated in FIG. 11 or 13, the irregularities may form a plurality of grooves. The height of the protruding objects needs not to be the same; the protruding objects are preferably configured so that an affected part can easily be rested on the area in which the irregularities are formed.

It is preferable that the area of contact between the affected part and sheet-like member be reduced by forming irregularities in the affected part resting area. In other words, it is preferable that the affected part cannot easily be brought into close contact with the sheet-like member. As a result of prevention of close contact between an affected part and the sheet-like member, heat is less likely to persist in the affected part.

As described above, the affected part resting area may be set in the first surface. It is preferable that the density of light irradiation from the surface light source to the affected part resting area of the first surface be higher than the density of light irradiation from the surface light source to another area of the first surface. Consequently, the therapeutic light availability is increased.

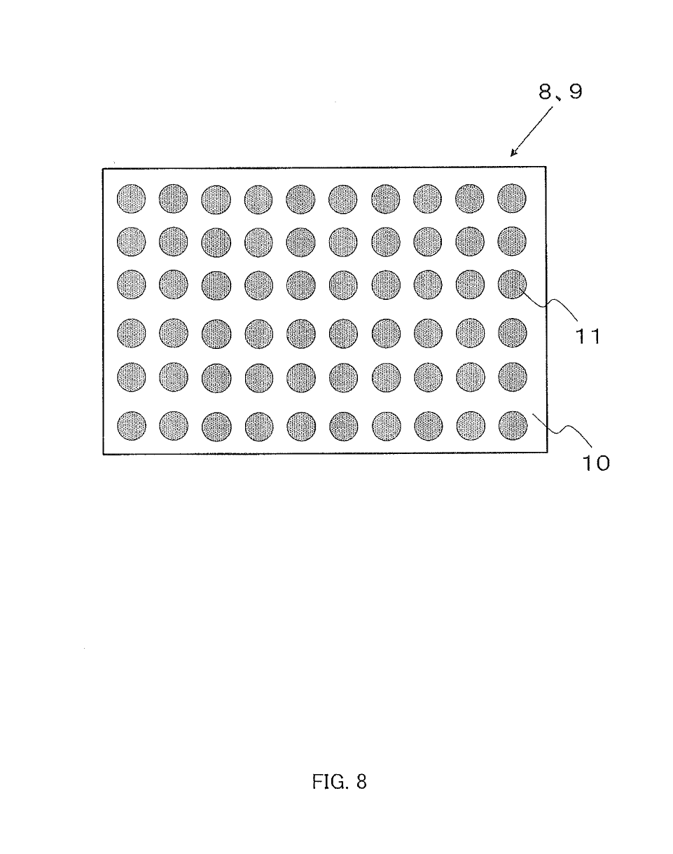

The surface light source may be composed of a single light source with the light source itself having a sheet-like shape or may be composed of a plurality of light-emitting devices disposed on a substrate (see, e.g., FIG. 8). When a light source composed of a plurality of light-emitting devices disposed on a substrate is used as the surface light source, the output of light from the surface light source can arbitrarily be controlled by adjusting the arrangement density of the light-emitting devices and/or direction of light irradiation from the respective light-emitting devices, allowing a desired area to have an increased light irradiation density.

The surface light source may be disposed so as to face the first surface of the sheet-like member or may be disposed so as to face the second surface on the opposite side of the first surface, or may be disposed on each of both of the first and second surfaces. If the sheet-like member is made of a material that transmits light from the surface light source(s), light from the surface light source disposed so as to face the second surface on the opposite side of the first surface on which an affected part is to be rested can be applied to the affected part rested on the first surface.

As described above, since the irregularities are formed at least in the affected part resting area of the first surface, an affected part does not easily come into close contact with the sheet-like member. Therefore, even if light from the surface light source disposed so as to face the second surface is applied to the affected part resting area, the temperature of the affected part resting area does not easily increase, and as a result, the temperature of the affected part does not excessively increase. On the other hand, if the affected part resting area is flat without any irregularities, the affected part comes into close contact with the sheet-like member. In such a case, if light from the surface light source disposed so as to face the second surface is applied to the affected part resting area, the temperature of the affected part resting area easily increases. As a result, the temperature of the affected part excessively increases. Therefore, the affected part may get burned or may sweat.

The surface light source may also be configured so that a light density in the affected part resting area is higher than a light density in an area of the first surface other than the affected part resting area. Consequently, the phototherapeutic efficiency is enhanced. In order to enhance the light density in the affected part resting area, light from the surface light source may intensively be applied to the affected part resting area.

It is preferable that light from the surface light source be light with a main wavelength in the infrared wavelength range. Infrared light can efficiently warm an affected part. In particular, in the case of a phototherapy apparatus for treating rheumatoid arthritis, it is preferable that light from the surface light source contains near-infrared light as the main component. Near-infrared light refers to light with wavelengths of around 700 nm to 2 μm. Among light of such wavelengths, infrared light with a wavelength of 750 to 900 nm is less absorbable by water and hemoglobin and thus is particularly suitable for treatment of rheumatoid arthritis.

The phototherapy apparatus according to the present invention may include a cooling fan. The cooling fan is preferably disposed so that air from the cooling fan flows on the first surface of the sheet-like member, preferably, the affected part resting area of the first surface. More specifically, the cooling fan can be disposed so that air from the cooling fan flows along the grooves defined by the irregularities on the first surface. In other words, it is preferable that the direction of air flow from the cooling fan and the direction in which the grooves extend are the same. Consequently, excessive heating of the affected part subjected to phototherapy is limited.

The phototherapy apparatus according to the present invention may be used for phototherapy of various types of symptom. For example, the phototherapy apparatus according to the present invention may be employed for, e.g., relief of pain due to chronic non-infectious inflammation of a muscle or a joint or treatment of rheumatoid arthritis. Also, although an affected part to be treated by the phototherapy apparatus according to the present invention is not specifically limited, the affected part may be an affected part in the vicinity of a joint such as a hand, a wrist, a foot, an ankle or a knee. The phototherapy apparatus according to the present invention is preferably used for phototherapy of rheumatoid arthritis.

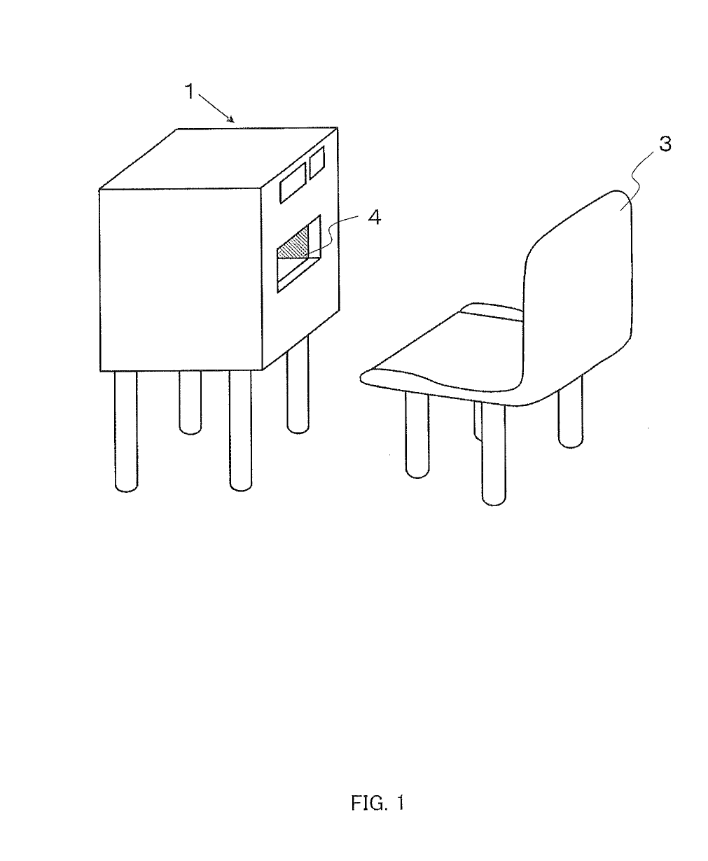

The phototherapy apparatus according to the present invention may include an enclosure that encloses the sheet-like member and the surface light source and includes an insertion opening for inserting an affected part to the inside thereof (see FIGS. 1 to 6). This is because, if light from the surface light source directly enters the eyes of a patient, the eyes may be damaged.

Embodiments of a phototherapy apparatus according to the present invention will be described in detail below with reference to the accompanying drawings; however, modes of a phototherapy apparatus according to the present invention are not limited by these embodiments.

### Embodiment 1

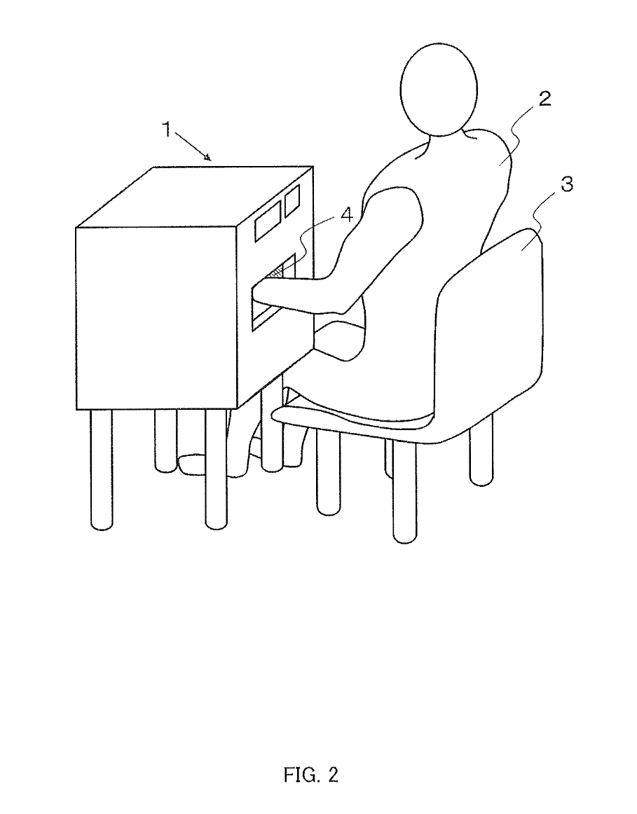

FIG. 1 is a perspective view of phototherapy apparatus 1 according to an embodiment of the present invention. As illustrated in FIG. 2, patient 2 sits on chair 3, and inserts an affected part into phototherapy apparatus 1 via insertion opening 4 for phototherapy. In Embodiment 1, treatment of a hand with rheumatoid arthritis is given as an example. Therefore, the affected part to be inserted into phototherapy apparatus 1 via insertion opening 4 is a hand.

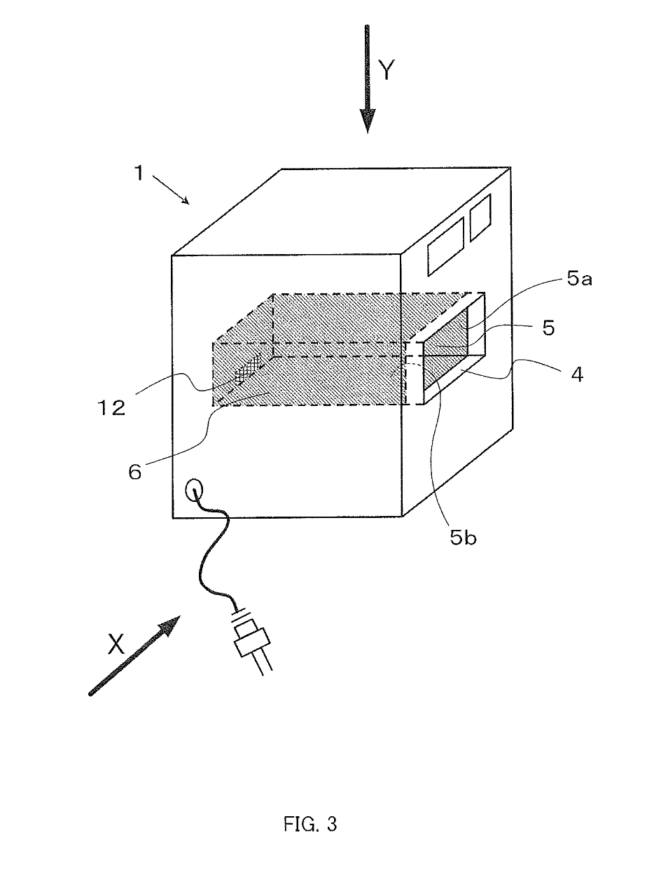

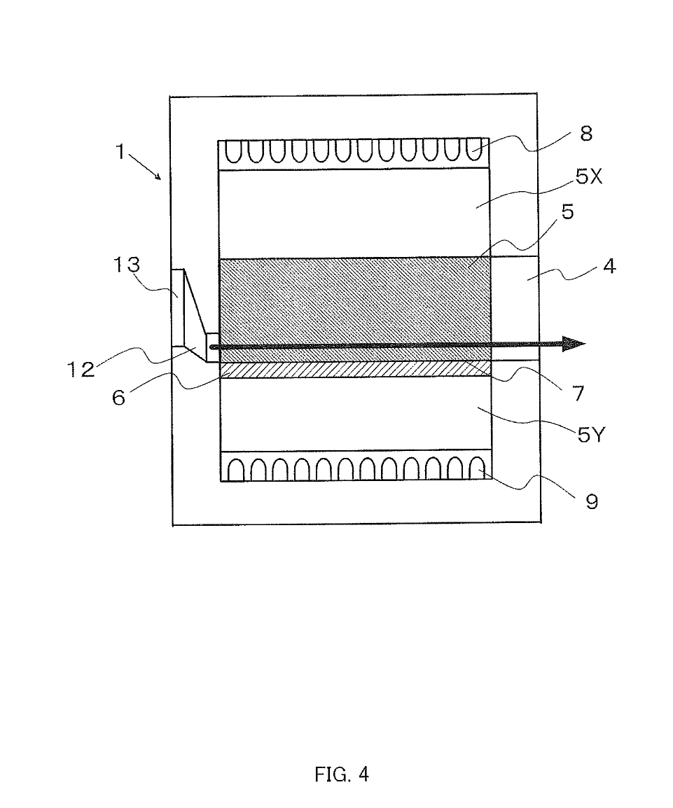

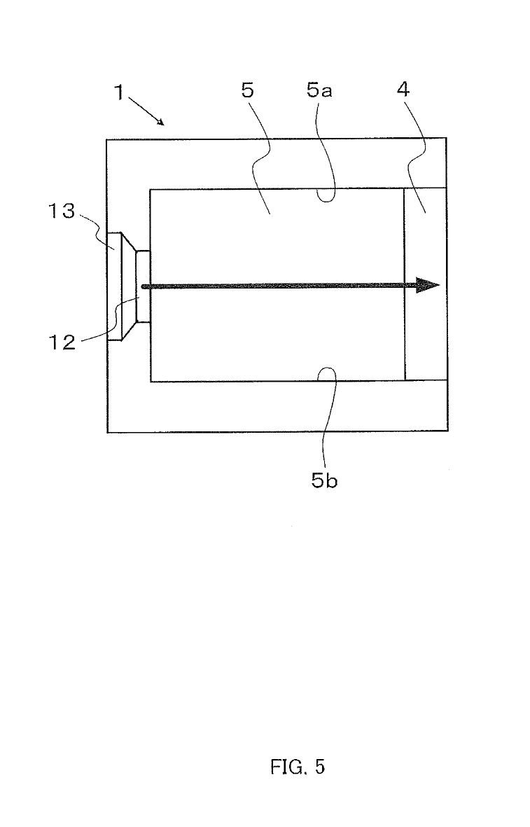

As illustrated in FIGS. 3 to 5, phototherapy apparatus 1 includes affected part insertion room 5 having a space that allows a hand to be inserted to the inside. FIG. 3 is a perspective view of phototherapy apparatus 1, which is a diagram in which the inside of phototherapy apparatus 1 can be seen through. FIG. 4 is a cross-sectional view of phototherapy apparatus 1 viewed along X in FIG. 3. FIG. 5 is a cross-sectional view of phototherapy apparatus 1 viewed along arrow Y in FIG. 3.

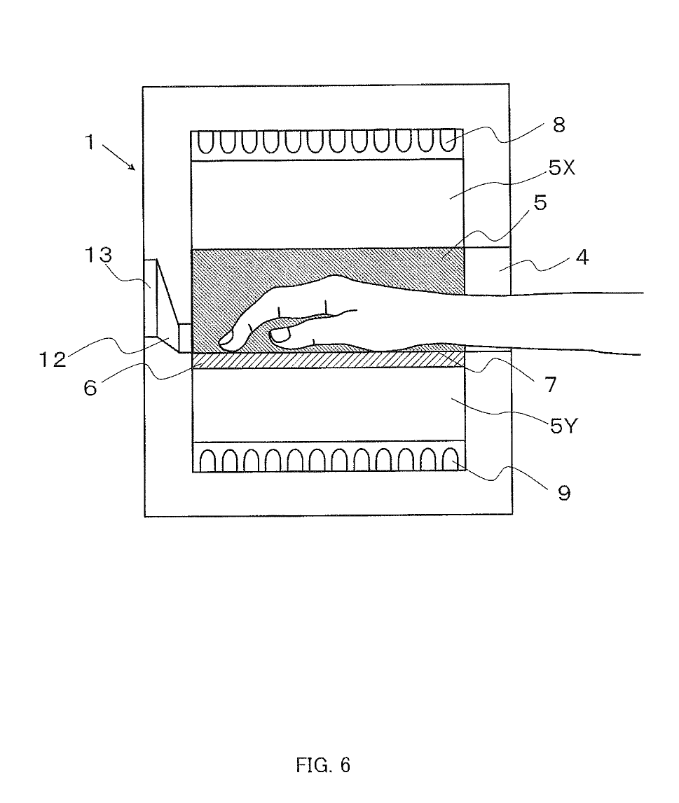

As in FIG. 4, FIG. 6 is a side cross-sectional view of phototherapy apparatus 1, which indicates a state in which a hand is inserted into affected part insertion room 5 via insertion opening 4. As described above, phototherapy apparatus 1 is configured so that an affected part is inserted into the inside thereof for treatment. Therapeutic light for treatment of rheumatoid arthritis contains invisible near-infrared light as a main component, and an output thereof is high. Therefore, as in the phototherapy apparatus according to Embodiment 1, a configuration is preferred that prevents possible impingement of therapeutic light into the eyes.

Near-infrared light is mainly used in treatment of rheumatoid arthritis for the following reasons. In treatment of rheumatoid arthritis, a sufficient therapeutic effect cannot actually be obtained unless light is delivered to a deep tissue in an affected part (for example, a joint in a hand, a foot or a finger). Mid- and far-infrared light is easily absorbed by water, and thus, is absorbed by, e.g., interstitial fluid in cells in the surface of the body. Therefore, mid- and far-infrared light is less likely to reach a deep tissue, and thus is unsuitable for phototherapy of rheumatoid arthritis. Also, visible light in a red range is absorbed by hemoglobin in blood. Therefore, visible light in the red range is unsuitable for phototherapy of rheumatoid arthritis because the therapeutic light is less likely to reach a deep tissue. On the other hand, near-infrared light exhibits excellent tissue permeability, and thus enables therapeutic light to be delivered to a deep tissue, and is suitable for treatment of rheumatoid arthritis.

As illustrated in FIG. 6, flat plate-like, sheet-like member 6 is fixated to affected part insertion room 5. More specifically, sheet-like member 6 is placed so as to extend from the lower edge of insertion opening 5 to the back of affected part insertion room 5. Affected part insertion room 5 is configured so that a hand inserted via insertion opening 4 can be rested on sheet-like member 6. Hereinafter, a surface of sheet-like member 6 on which a hand, an affected part, is to be rested is referred to as a first surface (affected part resting surface 7).

Sheet-like member 6 is made of a material that transmits light emitted from later-described light-emitting device 11. For example, sheet-like member 6 is made of hard material such as transparent acrylic resin or transparent glass.

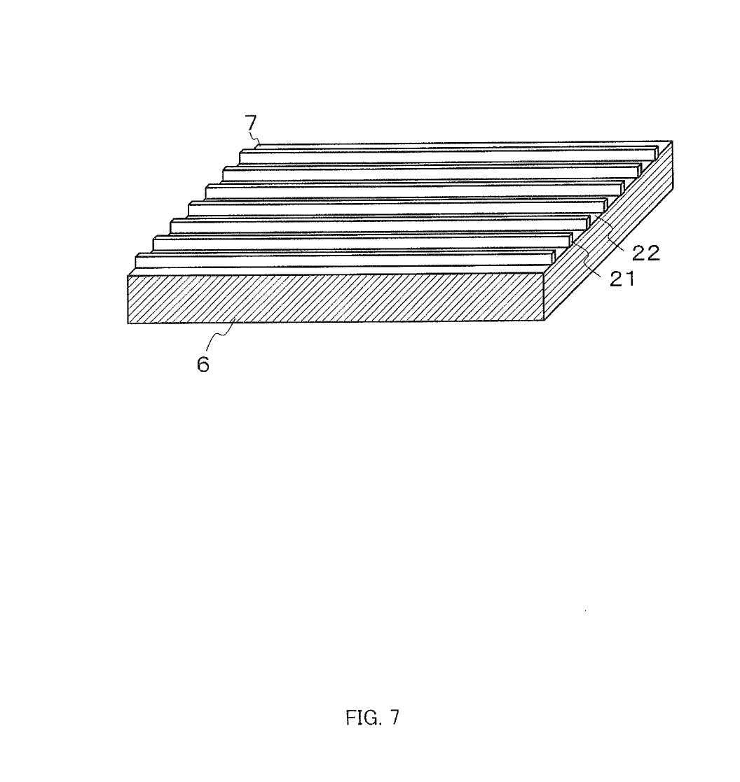

As illustrated in FIG. 7, the first surface (affected part resting surface 7) of sheet-like member 6 is formed so as to have irregularities thereon, thus forming protrusions 21. Protrusions 21 define a plurality of grooves 22 in the first surface (affected part resting surface 7), and grooves 22 extend from insertion opening 4 toward the back side.

In Embodiment 1, protrusions 21 formed in the first surface (affected part resting surface 7) define a plurality of grooves 22 that extend in one direction from insertion opening 4 to the back of affected part insertion room 5; however, the configuration of the protrusions formed in the first surface (affected part resting surface 7) in the present invention is not limited to the above one. In other words, any configuration can be employed as long as such configuration realize a reduced area of contact between a hand, an affected part, and the first surface (affected part resting surface) when the hand is rested on the protrusions and also enables heat persisting in the palm of the hand to be released through the grooves defined by the protrusions.

As illustrated in FIG. 4, first surface light source 8 is disposed with a certain space from the first surface (affected part resting surface 7) of sheet-like member 6 so as to face the first surface, and second surface light source 9 is disposed with a certain space from a second surface, which is a surface opposite to the first surface (affected part resting surface 7) of sheet-like member 6, so as to face the second surface. Each of first surface light source 8 and second surface light source 9 is configured to apply therapeutic light toward the first surface (affected part resting surface 7).

Space 5X is provided between affected part insertion room 5 and surface light source 8. Also, space 5Y is provided between sheet-like member 6 and surface light source 9. In Embodiment 1, the distance between the first surface (affected part resting surface 7) and first surface light source 8 is 20 cm and the distance between the first surface (affected part resting surface 7) and second surface light source 9 is 10 cm. The above distances are mere examples and the distances can freely be set according to the output level of light to be applied to affected part resting surface 7. If these distances are too small, unevenness of light to be applied to an affected part may easily occur.

Exemplary configurations of first surface light source 8 and second surface light source 9 will now be described. As illustrated in FIG. 8, each of first surface light source 8 and second surface light source 9 includes substrate 10, and multiple light-emitting devices 11 disposed on a surface of substrate 10. It is preferable that the surface of substrate 10 having light-emitting devices 11 disposed thereon be a reflective surface.

It is preferable that side wall surfaces 5a and 5b (see FIGS. 3 and 5) of affected part insertion room 5 be reflective surfaces. Also, it is preferable that inner surfaces of space 5X between affected part insertion room 5 and surface light source 8 and inner surfaces of space 5Y between sheet-like member 6 and surface light source 9 be reflective surfaces. Therefore, the inner surfaces of affected part insertion room 5 provide a kaleidoscope jointly with the surface of substrate 10. Light emitted from light-emitting devices 11 in each of first surface light source 8 and second surface light source 9 emit is applied to the first surface (affected part resting surface 7) and reflected by the reflective surfaces forming affected part insertion room 5, and thereby applied to an entire hand, an affected part. Consequently, therapeutic light can evenly be applied to all surfaces, including the upper side (back) and the lower side (palm), of a hand, an affected part.

Since Embodiment 1 is directed to a configuration of a phototherapy apparatus for treating rheumatoid arthritis, light-emitting devices 11 output therapeutic light including near-infrared light, a light considered as being optimal for treatment of rheumatoid arthritis, as the main wavelength band. Also, since Embodiment 1 is intended for treatment of rheumatoid arthritis, the phototherapy apparatus is configured so that the output level of light to be applied by each of first surface light source 8 and second surface light source 9 to affected part resting surface 7 is 115 mW/cm². The output level of each surface light source can be adjusted depending on the use of the phototherapy apparatus.

As illustrated in FIGS. 4 and 5, air inlet 12 that allows the inside of affected part insertion room 5 and the outside of phototherapy apparatus 1 to be in communication with each other is provided in a wall surface on the outer side of the back of affected part insertion room 5 relative to insertion opening 4. Also, cooling fan 13 is provided at a wall on the outer side of air inlet 12 in phototherapy apparatus 1.

Cooling fan 13 sends air external to the apparatus into affected part insertion room 5 through air inlet 12, and sends air inside affected part insertion room 5 to the outside of the apparatus through insertion opening 4. In other words, cooling fan 13 is configured so as to send air in the direction indicated by arrow in each of FIGS. 4 and 5.

As described above, protrusions 21 formed in the first surface (affected part resting surface 7) of sheet-like member 6 define a plurality of grooves 22 that extend in one direction from the entrance toward the back of affected part insertion room 5. Accordingly, the direction of air flow provided by cooling fan 13 (arrow direction indicated in each of FIGS. 4 and 5) and the direction in which grooves 22 extend are the same. In other words, a configuration is provided wherein air is sent along the direction in which grooves 22 extend.

The foregoing configuration limits an increase in the internal temperature of the affected part insertion room 5 due to irradiation of therapeutic light emitted from first surface light source 8 and second surface light source 9, as well as an increase in the skin temperature of hand, an affected part. In particular, air sent from cooling fan 13 flows through grooves 22, whereby heat generated in the affected part (palm of the hand) by light from second surface light source 9 is released. Thus, an increase in the skin temperature of the affected part (palm of a hand) can easily be limited and, e.g., sweating of the palm of the hand can be limited.

One of the objects of the present invention is to limit an excessive increase in the skin temperature of the affected part brought into contact with the sheet-like member. Accordingly, although phototherapy apparatus 1 according to Embodiment 1 includes first surface light source 8 and second surface light source 9, an object of the present invention can be achieved by second surface light source 9 alone from among the first surface light source and the second surface light source. In other words, the first surface light source is not an essential component.

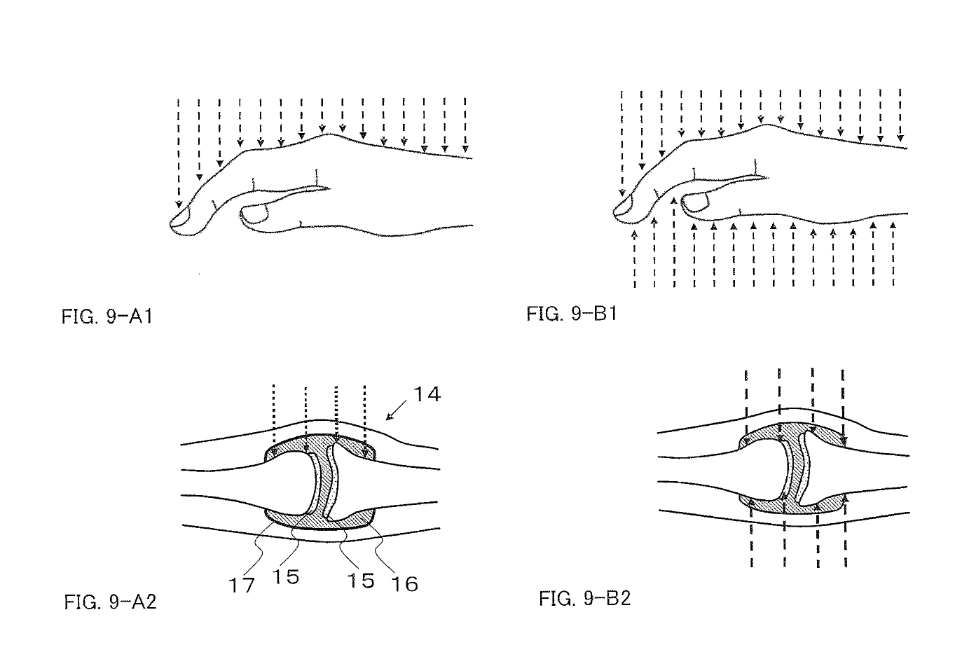

However, treatment of rheumatoid arthritis may require long-time irradiation using high-power light. In such a treatment, it is also necessary that sufficient therapeutic light be delivered to a deep tissue while limiting an increase in skin temperature. Therefore, it is preferable a phototherapy apparatus according to the present invention include a first surface light source and a second surface light source. Reasons for this will be described below with reference to FIGS. 9-A1, 9-A2, 9-B1 and 9-B2.

FIGS. 9-A1 and 9-A2 illustrate a manner in which phototherapy of a hand, an affected part, is performed using a conventional surface light source. FIGS. 9-B1 and 9-B2 are diagrams illustrating a manner in which phototherapy of a hand, an affected part, is performed using the phototherapy apparatus according to Embodiment 1. Dashed arrows indicated in each of FIGS. 9-A1 and 9-A2 and FIGS. 9-B1 and 9-B2 indicate therapeutic light applied by surface light source(s). In FIGS. 9-A1 and 9-A2, light is applied to the back of a hand, an affected part, from the upper side. In FIGS. 9-B1 and 9-B2, light from first surface light source 8 is applied to the back of a hand, an affected part, from the upper side and light from second surface light source 9 is applied to the palm of the hand from the lower side.

FIGS. 9-A2 and 9-B2 are transparent diagrams with an enlargement of a phalangeal joint portion of a hand that is an affected part to be subjected to phototherapy. A periphery of articular cartilage 15 in phalangeal joint 14 is filled with synovial fluid 16. Articular cartilage 15 and synovial fluid 16 are covered by synovial membrane 17. Rheumatoid arthritis is a disease in which synovial membrane 17, a deep tissue, inflames. Thus, in phototherapy, it is necessary to supply sufficient therapeutic light to synovial membrane 17, a deep tissue in phalangeal joint 14.

In the conventional configuration illustrated in FIGS. 9-A1 and 9-A2, therapeutic light from the surface light source is applied to the back of the hand from the upper side. Thus, in order to supply sufficient light to synovial membrane 17 positioned on the palm side, it is necessary to supply very high-power therapeutic light. This greatly increases the temperature of the skin surface of the back of the hand to which the therapeutic light from the surface light source is directly applied. As a result, redness, or a low temperature burn depending on the length of time of the treatment, may occur at the skin surface of the back of the hand. In particular, if the back of the hand has something that easily absorbs light such as a mole, the risks of burn and the like increase.

As described above, in the conventional configuration indicated in FIGS. 9-A1 and 9-A2, sufficient therapeutic light can be supplied to synovial membrane 17 positioned on the back side of the hand, but no sufficient light can be supplied to synovial membrane 17 positioned on the palm side of the hand because of e.g., existence of the bones that block the light. It may be possible to supply light to synovial membrane 17 by increasing the output of the therapeutic light; however, the back and the palm of the hand are unevenly treated.

In general, it is said that if the surface of a skin of a human body is continuously heated at 44° C. for approximately 360 minutes, the risk of a low temperature burn occurs. Then, it is said that the risk of a low temperature burn occurs in a half of the heating time for every temperature increase of 1° C. In other words, the risk of a low temperature burn occurs in heating time of approximately 180 minutes at 45° C., approximately 90 minutes at 46° C. and approximately six minutes at 50° C. As described above, the conventional rheumatoid arthritis treatment using high-power light involves the risk of, e.g., a low temperature burn.

On the other hand, in the configuration of the present invention indicated in FIGS. 9-B1 and 9-B2, therapeutic light is applied to the back of a hand from the upper side and therapeutic light is applied to the palm of the hand from the lower side. With this configuration, it is sufficient that the therapeutic light applied to the back of the hand and the therapeutic light applied to the palm of the hand have a level of power that can penetrate to synovial membrane 17 on the back side of the hand and synovial membrane 17 on the palm side of the hand, respectively. Consequently, a sufficient therapeutic effect can be attained.

As described above, the configuration of the present invention indicated in FIGS. 9-B1 and 9-B2 eliminates the need to supply very high-power therapeutic light as opposed to the conventional configuration indicated in FIGS. 9-A1 and 9-A2. As a result, the risk of, e.g., a burn of a skin surface to which therapeutic light is directly applied can substantially be decreased. Also, the configuration of the present invention enables even supply of light to an entire affected part and thus limits unevenness of therapeutic light to be supplied to the back side and the palm side of a hand. In other words, proper treatment can be performed.

However, in the configuration of the present invention indicated in FIGS. 9-B1 and 9-B2, heat caused by light from second surface light source 9 is likely to persist in the palm of a hand, an affected part, rested on the affected part resting surface. Therefore, as indicated in Embodiment 1, protrusions 21 are formed in affected part resting surface 7 of sheet-like member 6 to provide grooves 22 thus limiting an increase in the skin temperature of the palm of a hand, an affected part.

### Embodiment 2

Since Embodiment 2 has a basic configuration that is the same as that of phototherapy apparatus 1 according to Embodiment 1, only parts that are different from those of Embodiment 1 will be described and a description of parts that are the same as those of Embodiment 1 will be omitted.

In Embodiment 1, protrusions 21 are formed over the entire first surface (affected part resting surface 7) of the sheet-like member, providing grooves 22. In Embodiment 2, affected part resting area 18 on which an affected part to be treated is rested is set in a first surface (affected part resting surface 7) and a plurality of protrusions 19 are formed in affected part resting area 18, whereby grooves are provided.

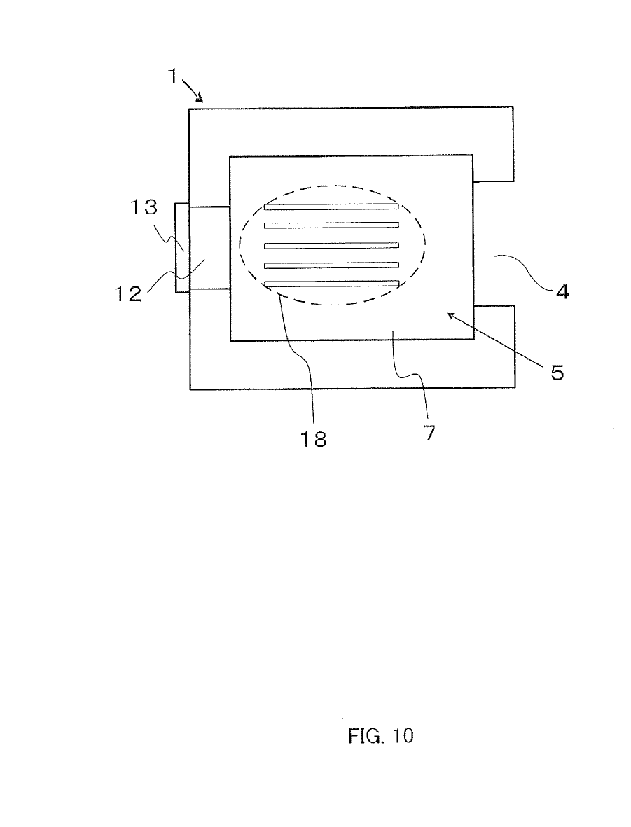

FIG. 10 is a top view of affected part insertion room 5 in Embodiment 2. As can be understood from FIG. 10, affected part resting area 18 (area surrounded by the dotted line) is set in a substantially central part of affected part resting surface 7, and as illustrated in FIG. 11, a plurality of protrusions 19 (five protrusions 19 in Embodiment 2) are disposed in affected part resting area 18. Protrusions 19 are disposed in affected part resting area 18 at predetermined intervals 20 thus providing grooves.

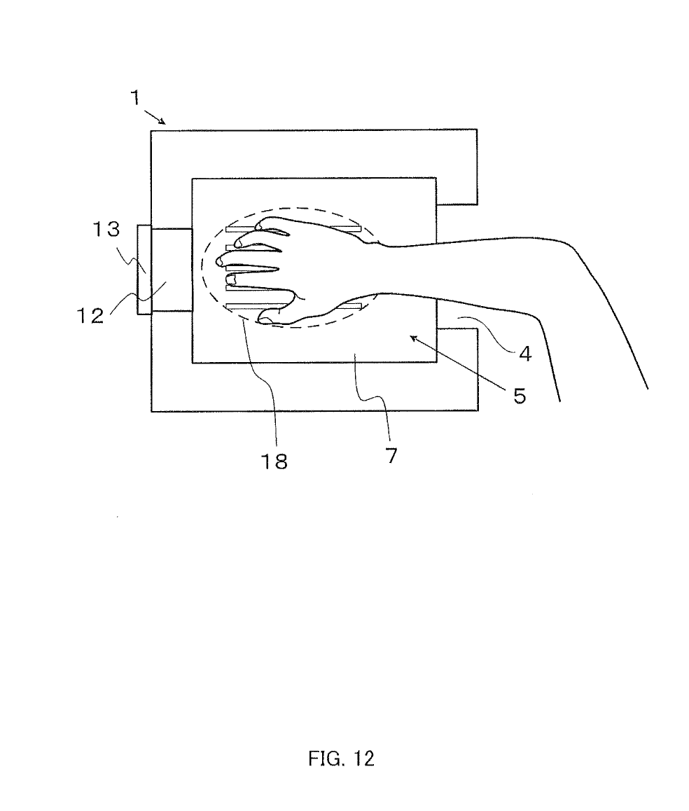

As illustrated in FIG. 12, a hand, an affected part, is rested on affected part resting area 18. Consequently, the area of close contact between the palm of a hand, an affected part, and affected part resting surface 7 of a sheet-like member is reduced to limit persistence of heat in the palm of the hand. Accordingly, redness or a low temperature burn of the skin surface of the palm of the hand is limited.

Furthermore, as illustrated in FIG. 10, air is sent to affected part insertion room 5 via air inlet 12, and air inside affected part insertion room 5 is discharged via insertion opening 4, by cooling fan 13. The direction of the air flow and the direction in which the grooves defined by the plurality of protrusions 19 extends are the same, whereby persistent of heat in the palm of a hand, an affected part, rested on affected part resting area 18 is limited. Consequently, the risks of burn and the like can further be reduced.

Also, protrusion 19 is made of a material that transmits light emitted from light-emitting devices 11, enabling mainly light from second surface light source 9 to be efficiently applied to the affected part. Consequently, the phototherapeutic efficiency is enhanced.

Also, in the present embodiment, the position where an affected part is rested is defined as affected part resting area 18. Therefore, light that is not likely to contribute to treatment (i.e., light to be applied to a part other than the affected part) can be reduced by enhancing the density of light from each of first surface light source 8 and second surface light source 9 in affected part resting area 18. In other words, phototherapy with high light use efficiency can be achieved. In order to enhance the light density in affected part resting area 18, for example, the main light emission axes of the respective light-emitting devices 11 included in each of first surface light source 8 and second surface light source 9 may be directed to affected part resting area 18.

Embodiment 2 is directed to a configuration wherein sufficient light can be delivered to a deep tissue while an increase in the skin temperature of the affected part due to irradiation of light to the affected part is further limited.

Although Embodiment 2 is directed to a configuration wherein flat plate-like equally-sized protrusions 19 are disposed on the first surface (affected part resting surface 7) of the sheet-like member, the configuration of protrusions 19 is not limited to such one.

For example, as illustrated in FIG. 13, protrusions 19 may be of different size so that a surface defined by the respective top surfaces of protrusions 19 disposed in affected part resting area 18 forms a curved surface as a whole. A patient that suffers rheumatoid arthritis on his/her hand gets, e.g., a pain when he/she moves the hand, in particular, the phalangeal joints. Therefore, the surface formed by the top surfaces of protrusions 19 is curved so as to fit the palm of the hand. As a result, a patient can easily rest his/her hand on affected part resting area 18.

Although Embodiment 2 is directed to a configuration wherein a plurality of flat plate-like members are used as protrusions 19, as illustrated in FIG. 14, a plurality of rod-like members may be used as protrusions 19. Consequently, the area of close contact between the affected part and sheet-like member can further be reduced. Therefore, heat is less likely to persist in the affected part, further reducing the risks of burn and the like.

### INDUSTRIAL APPLICABILITY

The phototherapy apparatus according to the present invention is configured as described above, enabling sufficient treatment to be performed by delivering sufficient light to a deep tissue while an increase in skin temperature is limited.

Thus, the phototherapy apparatus according to the present invention is expected to be widely used as a phalangeal joint phototherapy apparatus for treatment of joints with rheumatism.

### REFERENCE SIGNS LIST

1 Phototherapy apparatus
2 Patient
3 Chair
4 Insertion opening
5 Affected part insertion room
6 Sheet-like member
7 Affected part resting surface
8 First surface light source
9 Second surface light source
10 Substrate
11 Light-emitting device
12 Air inlet
13 Cooling fan
14 Phalangeal joint
15 Articular cartilage
16 Synovial fluid
17 Synovial membrane
18 Affected part resting area
19 Protrusion
20 Interval
21 Protrusion
22 Groove

1-10. (canceled)

11. A phototherapy apparatus comprising:
a sheet-like member including a first surface on which an affected part is to be rested; and
a surface light source configured to apply light toward the sheet-like member,
wherein an affected part resting area that defines a position where the affected part is rested is set in a part of the first surface, and
a plurality of grooves are provided in the affected part resisting area by forming thereon irregularities.

12. The phototherapy apparatus according to claim 11, wherein the surface light source is disposed at a position where light is applied toward the diseased part resting area.

13. The phototherapy apparatus according to claim 12, wherein the surface light source is configured so that a light density in the affected part resting area is higher than a light density in an area of the first surface other than the affected part resting area.

14. The phototherapy apparatus according to claim 11, wherein the sheet-like member is made of a material that transmits light there through.

15. The phototherapy apparatus according to claim 11, wherein the surface light source includes a surface light source disposed so as to face a second surface that is a surface opposite to the first surface.

16. The phototherapy apparatus according to claim 11, wherein the surface light source includes a surface light source disposed so as to face the first surface, and a surface light source disposed so as to face a second surface that is a surface opposite to the first surface.

17. The phototherapy apparatus according to claim 11, further comprising a cooling fan configured to cool the affected part rested on the first surface,
wherein the plurality of grooves are configured so as to pass air from the cooling fan therethrough.

18. The phototherapy apparatus according to claim 11, wherein the surface light source emits light with a main wavelength in an infrared wavelength range.

19. The phototherapy apparatus according to claim 11, wherein the phototherapy apparatus is configured to treat an affected part of a patient suffering from rheumatoid arthritis.
